(19) United States
(12) Patent Application Publication
(10) Pub. No.: US 2026/0348123 A1
(43) Pub. Date: Nov. 5, 2026

(54) RAPID PILE LOADING TEST METHOD UTILIZING CASE METHOD

(71) Applicant: JIBANSHIKENJO Co, Ltd., Tokyo (JP)

(72) Inventors: Isaku YAMAMOTO, Tokyo (JP); Shuichi KAMEI, Tokyo (JP); Tatsunori MATSUMOTO, Tokyo (JP); Atsushi NAKAYAMA, Tokyo (JP); Shihchun LIN, Tokyo (JP)

(73) Assignee: JIBANSHIKENJO Co, Ltd., Tokyo (JP)

(21) Appl. No.: 19/472,889

(22) PCT Filed: Apr. 13, 2023
(86) PCT No.: PCT/JP2023/015051

§ 371 Date: Oct. 6, 2025

(51) Int. Cl. E02D 33/00

(52) U.S. Cl. CPC E02D 33/00

(57) ABSTRACT

The present invention is to provide a rapid load test method for a pile, with which a more highly precise load-displacement relation can be determined by use of a newly developed analysis method, on the premise that a plurality of rapid load tests shall be conducted on the pile head. The pile is subjected to a plurality of rapid load tests, and then, a w-Rₛₒᵢₗ relation is determined from both the soil resistance Rₛₒᵢₗ obtained from a Case method used for a conventional impulsive load test and the temporal changes of a directly measured pile displacement w to obtain an unloading point load R_{ULP} and an unloading point displacement from the determined w-Rₛₒᵢₗ relation. After that, two or more unloading points are connected; likewise, an Unloading Point Connection (ULPC) method is used for a conventional rapid load test, with the result that a static load-displacement relation can be finally determined.

### TECHNICAL FIELD

The present invention relates to a rapid load test method for a pile, in which a weight is dropped onto a pile head to determine a static load-displacement relation of the pile from a relation between a load caused by the drop of the weight and a displacement of the pile head. More specifically, the present invention relates to a rapid load test method for a pile, with which a more highly precise load-displacement relation can be determined by use of a newly developed analysis method, when a plurality of rapid load tests are conducted on a pile head in the manner of changing a weight drop height to determine a relation between a pile head load and a pile head displacement from the result of measurements by the rapid load test. This newly developed analysis method enables a rapid load test using a weight that is lighter than ever before.

### BACKGROUND ART

As some patent documents on a rapid load test method for a pile/an analysis method in the prior arts, the following patent documents 1 to 3 can be given, for instance.

In relation to a rapid load test method for determining pile settlement stiffness and pile bearing capacity by dropping a weight onto a pile head to apply striking force to the pile head, the patent document 1 (Japanese Unexamined Patent Application Publication No. 2002-303570) discloses a technique that makes it possible to obtain a highly precise test result with a smaller load in such a way that a cushion material made of a material with specific gravity in the range of not less than 0.35 to not more than 0.5 is interposed between the weight and the pile head.

As one rapid load test equipment for a pile for obtaining highly reliable data simply in a short period of time, as data necessary for estimation of bearing capacity of the pile driven into the ground, the patent document 2 (Japanese Unexamined Patent Application Publication No. 2005-068802) discloses the rapid load test equipment for a pile, in which a cushion material formed by laminating two or more metal plates and a polymer material sandwiched between these metal plates is interposed between a pile head and a weight for applying a blow to the pile head.

The patent document 3 (Japanese Patent Publication No. 6613430) discloses the rapid load test equipment for a pile for obtaining highly reliable data simply in a short period of time in such a way that a weight dropped onto a pile head and subsequently bounding therefrom is made to quickly catch to discontinue rebounding of the weight, in cases where a cushion material is interposed between the pile head and the weight for applying a blow to the pile head.

In Japan, as to the rapid load test method for the pile as described above, it has been 20 years since rapid load testing on piles was incorporated, as one dynamic load test method, in the rapid load testing standards (JGS1815-2002) of the Japanese Geotechnical Society. As of 2002 when the rapid load testing was incorporated in the rapid load testing standards, an inertial force method using a reaction body had been dominantly adopted for the force application equipment used for rapid load tests, whereas as of this moment with advanced improvements on the force application equipment, most of rapid load tests are being conducted with a weight drop method using a soft cushion. Accordingly, in regards to the number of times of loading to be performed, there has been a tendency to adopt loading that is performed a plurality of times in the manner of heightening a hammer drop height in steps such as to finally load a load equivalent to the ultimate bearing capacity, in place of a loading method in which loading of the load equivalent to the ultimate bearing capacity is performed once.

For the inertial force method using the reaction body, an Unloading Point (ULP) method has been applied to determination of a load-displacement relation from dynamic signals resulting from measurements in the loading performed once (See FIG. 1). By contrast with the above method, the adoption of the loading performed a plurality of times has resulted in that both of an unloading point load regarded equal with static resistance and a pile displacement at that moment can be obtained the same number of times of dropping. Therefore, an Unloading Point Connection (ULPC) method of simply connecting a load and a displacement at two or more unloading points has been taking the lead in determining the load-displacement relation of the pile (See FIG. 2).

### [Unloading Point (ULP) Method]

For the Unloading Point method, the effect of wave propagation in a pile body is assumed to be negligible for the rapid load test on the pile. Meanwhile, as to the rapid load test, the pile is assumed to be a point mass of a rigid body, so that static soil resistance R_{w}(t) and dynamic soil resistance Rᵥ(t) are considered as acting on the pile when dynamic load F_{rapid}(t) acts on the pile.

The relation between these is given by the following equations (1) and (2), and also as shown in FIG. 1.

[Equation 1]

Rₛₒᵢₗ(t)=F_{rapid}(t)−Rₐ(t)=F_{rapid}(t)−m·α(t) (1)

[Equation 2]

Rₛₒᵢₗ(t)=R_{w}(t)+Rᵥ(t)=R_{w}(t)+C·v(t) (2)

F_{rapid} is the load in the pile head, Rₛₒᵢₗ is soil resistance, Rₐ is the inertial force of the pile body, R_{w} is the static soil resistance component, Rᵥ is dynamic soil resistance component, m is mass of the pile body, a is acceleration of the pile body, C is damping constant, and v is velocity of the pile body.

The maximum value wₘₐₓ of a displacement w is called an unloading point. At the unloading point, the pile velocity v is 0, and as a result, the load (or unloading point load R_{ULP}) at the unloading point is considered as being equal to the static soil resistance R_{W}.

To determine the static soil resistance R_{W} at points other than the unloading point, it is necessary to determine the damping constant C. It is noted that non-patent document 1 (Kusakabe O., Matsumoto T. (1995): Statnamic tests of Shonan test program with review of signal interpretation, Proc. 1st Int. Statnamic Seminar, Vancouver, Canada, pp. 113-122) suggests that the damping constant C should be determined by the following equation (3).

[Equation 3]

C=(Rₛₒᵢₗ₍ₘₐₓ₎−Rᵤₗₚ)/v* (3)

Rₛₒᵢₗ₍ₘₐₓ₎ is maximum value of Rₛₒᵢₗ, and v* is pile displacement velocity as of Rₛₒᵢₗ₍ₘₐₓ₎.

Assuming that the damping constant C shall remain unchanged irrespective of the pile displacement and/or pile velocity, a R_{w}−w relation can be determined using the following equation (4).

[Equation 4]

R_{w}(t)=Rₛₒᵢₗ(t)−C·v(t) (4)

### [Unloading Point Connection (ULPC) Method]

The above Unloading Point (ULP) method supposes that the loading of the load equivalent to the ultimate bearing capacity shall be performed once, and hence, determination of the damping constant C is required for this Unloading Point method. In which case, however, there may be changes in the value of the damping constant C depending on the pile displacement and/or pile velocity.

For the load test conducted with the weight drop method using the soft cushion, the loading performed a plurality of times in the manner of heightening the hammer drop height in steps shall be adopted. Therefore, on the occasion of applying each blow, two or more unloading point loads are obtained together with two or more unloading point displacements corresponding thereto. Accordingly, the unloading points are simply connected, thereby allowing the R_{w}−w relation as shown in FIG. 2 to be simply obtained only by connecting the unloading points without any need to determine the damping constant C required for the Unloading Point (ULP) method.

However, for the Unloading Point (ULP) and Unloading Point Connection (ULPC) methods, the pile is assumed to be the point mass of the rigid body, and as a result, the effect of elastic deformation of the pile is made greater as the pile is larger in length. For instance, it happens that the result of the estimation on the load-displacement relation is affected by a pile acceleration measurement position.

### [Case Method Based on One-Dimensional Wave Propagation Theory]

The Case method, as described in non-patent document 2 (Raushe F. Goble G., Likins G. E. Jr. (1985): Dynamic determination of pile capacity, ASCE Jour. Geotech. Div., 111(3), pp. 367 to 383), is a method that has been used for an impulsive load test, more specifically, a method that is used to determine static penetration resistance based on one-dimensional wave propagation theory by use of both an empirical coefficient called the J_{c} coefficient and penetration resistance of the pile (soil resistance to the pile) upon receiving a blow. However, this method does not take the load-displacement relation determined with the rapid load test into consideration.

First of all, a downward traveling wave F_{d} and an upward traveling wave Fᵤ are calculated from measured signals (axial force F and pile velocity v) (See the following equations (5) and (6)). After that, temporal changes of the soil resistance Rₛₒᵢₗ to the pile are obtained using the following equation (7) (See FIG. 3).

$$\begin{matrix}[ Equation 5 ] & \\ {F}_{d} ( {x}_{m} , t ) = \frac{F ( {x}_{m} , t ) + Z v ( {x}_{m} , t )}{2} & ( 5 )\end{matrix}$$

$$\begin{matrix}[ Equation 6 ] & \\ {F}_{u} ( {x}_{m} , t ) = \frac{F ( {x}_{m} , t ) - Z v ( {x}_{m} , t )}{2} & ( 6 )\end{matrix}$$

$$\begin{matrix}[ Equation 7 ] & \\ {R}_{soil} ( {x}_{m} , t ) = {F}_{d} ( {x}_{m} , t - \frac{{L}_{m}}{c} ) + {F}_{u} ( {x}_{m} , t + \frac{{L}_{m}}{c} ) & ( 7 )\end{matrix}$$

Here, x is coordinate along a pile axis (with the pile head set to be 0), xₘ is measurement position, v is pile velocity, Lₘ is length from the measurement position to a pile tip, F is axial force, F_{d} is downward traveling wave, Fᵤ is upward traveling wave, Z is impedance (EA/c) of the pile body, c is longitudinal wave velocity, E is Young's modulus of a pile material, A is cross-sectional area of the pile, and Rₛₒᵢₗ is penetration resistance.

The Case method is aimed at evaluating the penetration resistance of the pile during driving, and therefore, any load-displacement relation of the pile cannot be obtained with the Case method alone. However, because of the fact that the Case method is a method based on one-dimensional wave propagation theory, the Case method can evaluate the penetration resistance of the pile almost correctly, even when the pile is larger in length.

Non-patent document 3 (Kojima, Nishimura (2021): Soil resistance analysis method in rapid load test, the 56th Japan National Conference on Geotechnical Engineering) describes the numerical analysis of a rapid load test on a pile for weighing soil resistance obtained from analysis with the Case method against that obtained from analysis with the Unloading Point (ULP) method. The result of weighing shows that when a relative loading time Tᵣ=tₗ/(2 Lₘ/c) (where ty is the loading duration) is below 5, the soil resistance obtained from the latter analysis overestimates the value obtained from the former analysis. However, this non-patent document 3 makes no mention of any effect on the load-displacement relation.

Non-patent document 4 (Matsuzawa, Nakashima, Nakayama, Matsumoto (2008): A piling method accompanying rapid load testing, Proc, the 8th Int. Conf. on the Application of Stress-Wave Theory to Piles, Lisbon, Portugal, pp. 487 to 495) describes a rapid load test on a tip bearing pile for determining a static load-displacement relation of the pile with the invocation of a Case method. For this test, a blow with a relatively large striking force has been applied once. For that reason, the determination of the damping constant C has been necessitated.

### SUMMARY

For the Unloading Point and Unloading Point Connection methods, the shorter the relative loading time Tᵣ is, the more inferior the analysis precision becomes. Accordingly, there have been such problems that the test equipment on a considerably large scale is needed to lengthen the relative loading time Tᵣ, and that much increase in the cost for the test conducted once is incurred as well.

An object of the present invention is to provide a rapid load test method for a pile, with which a more highly precious load-displacement relation can be determined by use of a newly developed analysis method in which the concept of a Case method used for an impulsive load test is incorporated in a conventional Unloading Point Connection (ULPC) method, on the premise that a rapid load tests shall be conducted on a pile head a plurality of times.

The present invention addresses the problem of providing a rapid load test method for a pile, in which a weight is dropped onto a pile head to determine a static load-displacement relation of the pile from a relation between a load caused by the drop of the weight and a displacement of the pile head, wherein the rapid load test method comprises the steps of:
conducting a plurality of rapid load tests on said pile head in the manner of changing the drop height h of said weight,
determining soil resistance Rₛₒᵢₗ to the pile under receiving a blow, in order of the following procedures (a) and (b) according to a Case method based on one-dimensional wave propagation theory, for each of the plurality of rapid load tests,
determining a relation between a displacement w of said pile head and said soil resistance Rₛₒᵢₗ from temporal changes of the directly measured displacement w of said pile head, determining an unloading point load Pₕ and an unloading point displacement wₕ by assuming that the soil resistance Rₛₒᵢₗ₍ₘₐₓ₎ as of the maximum displacement wₘₐₓ is being static resistance R_{w}, and
determining a pile head load Pₕ-pile head displacement wₕ relation resulting from connecting said unloading point load Pₕ and said unloading point displacement wₕ, respectively obtained for each different drop height h.

(a) Calculating a downward traveling wave F_{d} and an upward traveling wave Fᵤ from axial force F and pile velocity v in the rapid load test by using the following equations (5) and (6).

$$\begin{matrix}[ Equation 5 ] & \\ {F}_{d} ( {x}_{m} , t ) = \frac{F ( {x}_{m} , t ) + Z v ( {x}_{m} , t )}{2} & ( 5 )\end{matrix}$$

$$\begin{matrix}[ Equation 6 ] & \\ {F}_{u} ( {x}_{m} , t ) = \frac{F ( {x}_{m} , t ) - Z v ( {x}_{m} , t )}{2} & ( 6 )\end{matrix}$$

(b) Subsequently, obtaining the temporal changes of the soil resistance Rₛₒᵢₗ to the pile by using the following equation (7).

$$\begin{matrix}[ Equation 7 ] & \\ {R}_{soil} ( {x}_{m} , t ) = {F}_{d} ( {x}_{m} , t - \frac{{L}_{m}}{c} ) + {F}_{u} ( {x}_{m} , t + \frac{{L}_{m}}{c} ) & ( 7 )\end{matrix}$$

Where x is coordinate along a pile axis (with the pile head set to be 0), xₘ is measurement position, v is pile velocity, Lₘ is length from the measurement position to a pile tip, F is axial force, F_{d} is downward traveling wave, Fᵤ is upward traveling wave, Z is impedance (EA/c) of a pile body, c is longitudinal wave velocity, E is Young's modulus of a pile material, A is cross-sectional area of the pile, and Rₛₒᵢₗ is soil resistance.

Namely, for the present invention, a certain pile is subjected to application of a blow (the rapid load test) a plurality of times, and then, the w-Rₛₒᵢₗ relation is determined from both of the penetration resistance Rₛₒᵢₗ obtained from the Case method and the temporal changes of the directly measured displacement w of the pile to obtain the unloading point load R_{ULP} and the unloading point displacement from the determined w-Rₛₒᵢₗ relation. After that, two or more unloading points are connected, likewise the Unloading Point Connection (ULPC) method, with the result that the static load-displacement relation can be finally determined.

According to the rapid load test method for the pile in the present invention, it is possible to provide a rapid load test method for a pile, with which a more highly precise load-displacement relation can be determined by use of a newly developed analysis method in which the concept of the Case method used for the impulsive load test is incorporated in the conventional Unloading Point Connection (ULPC) method, on the premise that a rapid load test shall be conducted on the pile head more than once.

The rapid load test method for the pile in the present invention is considered to be based on one-dimensional wave propagation theory, and hence, can dispense with any inertial force correction for the pile. Accordingly, this rapid load test method provides the advantage that restrictions on application thereof due to the pile length or due to the relative loading time can be eased, for instance. Besides, in contrast to the conventional Unloading Point and Unloading Point Connection methods, which have had such problems that the test equipment on the considerably large scale is needed to lengthen the relative loading time Tᵣ and that much increase in the cost for the test conducted once is incurred as well, it is made possible for this rapid load test method to solve these problems.

### BRIEF DESCRIPTION OF DRAWINGS

FIG. 1 is a graphical representation of a load-displacement relation regarding an Unloading Point (ULP) method, given as one conventional rapid load test method.

FIG. 2 is a graphical representation of a load-displacement relation regarding an Unloading Point Connection (ULPC) method, given as another conventional rapid load test method.

FIG. 3 is a view for summarizing a Case method used for a conventional impulsive load test.

FIG. 4 is a view showing N-value distributions at a testing site and a state of pile penetration thereat.

FIG. 5 is a graphical representation of a pile head load Pₕ-pile head displacement w relation determined with the conventional Unloading Point (ULP) method.

FIG. 6 is a graphical representation of a pile head load Pₕ-pile head displacement w relation determined with the conventional Unloading Point Connection (ULCP) method.

FIG. 7 is a graphical representation of time histories of soil resistance and rapid load, and also those of displacement, velocity, and acceleration (where Tᵣ=3, and h=1.0 m).

FIG. 8 is a graphical representation of a pile head load Pₕ-pile head displacement w relation determined with a method (ULPC_CM method) according to the present invention.

FIG. 9 is a graphical representation of a comparison between the pile head load Pₕ-pile head displacement w relation determined with the conventional Unloading Point Connection (ULPC) method and that determined with the method (ULPC_CM method) according to the present invention.

FIG. 10 is a graphical representation of time histories of F_{rapid}, R_{soil(Case)}, and R_{soil(ULP)} in rapid load tests conducted respectively when Tᵣ=3, =4, and =5.

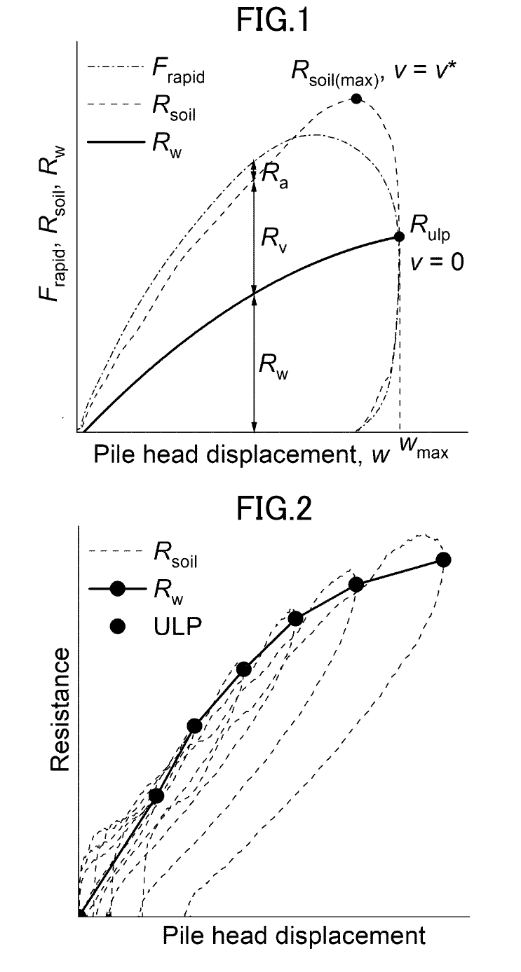

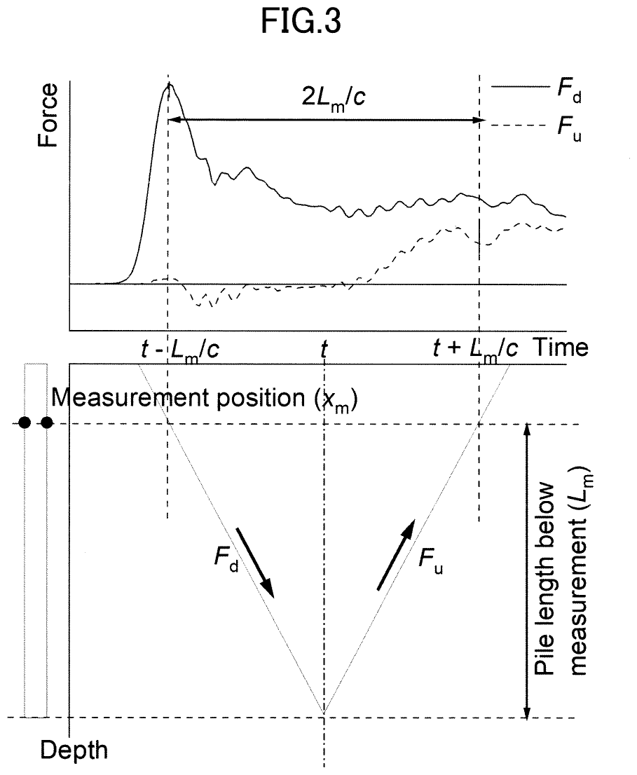

### DETAILED DESCRIPTION

Hereinafter, some tests conducted for the verification of the rationality of a rapid load test method according to the present invention will be described.

First of all, a pile load-pile displacement relation obtained from a static load test, that obtained from an Unloading Point (ULP) method, that obtained from an Unloading Point Connection (ULPC) method and that obtained from an analysis method (which will be referred to as “ULPC_CM method”) in the present invention were weighed against one another, under consideration of rapid load tests conducted respectively when the relative loading time Tᵣ=5, =4 and =3.

### [Test Pile Specification, Outline of Testing-Site Ground and Load Test Procedure]

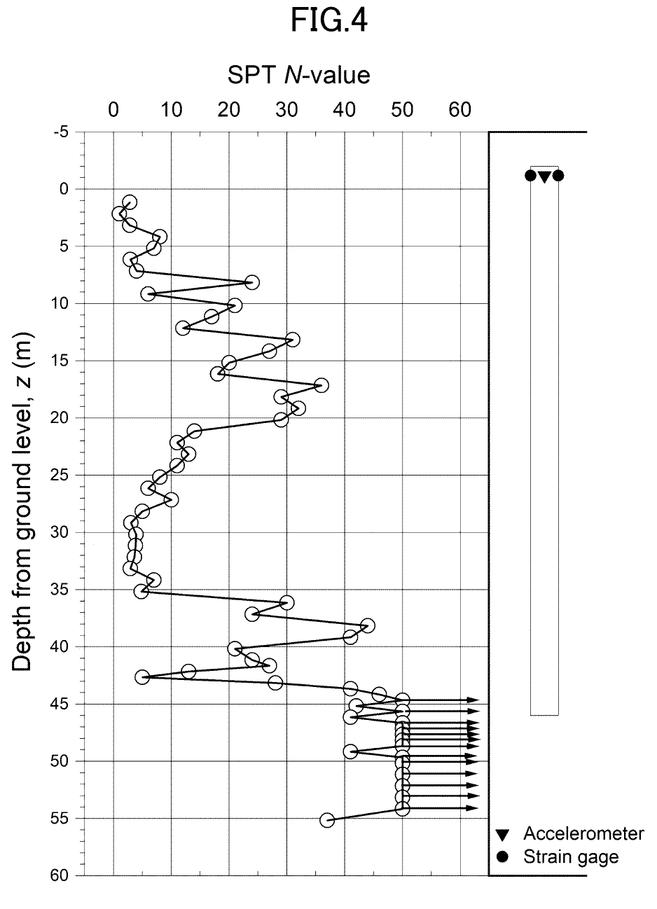

A steel-pipe test pile shown in Table 1 below was subjected to static load test (SLT) and also to rapid load test (RLT) using the rapid load test equipment (constituted such that a cushion material formed of a steel plate and elastomer is installed on a pile head) with adoption of a weight drop method using a soft cushion. The overall length of the test pile was 48.0 m, and penetration of this pile in a bearing stratum with an N-value of not less than 50 was carried out (see FIG. 4).

TABLE 1
Test pile specification

| Item | Value |
|---|---|
| Length, L(m) | 48.0 |
| Outer diameter, D₀ (mm) | 800 |
| Inner diameter, D₁ (mm) | 750 |
| Wall thickness, t_{w}(mm) | 25 |
| Cross-sectional area, A(m²) | 0.0609 |
| Young's modulus, E(kPa) | 2.00 × 10⁸ |
| Density, p(ton/m³) | 7.85 |
| Mass, m(ton) | 22.314 |
| Longitudinal wave velocity, c(m/s) | 5133 |

For the rapid load test, a weight with mass mₕ of 23 tons was used, and changes of the drop height h of the weight were made, ranging from 0.05 to 1.80 m. According to the rapid load test, the relative loading time Tᵣ was set to be Tᵣ=t_{L}/(2L/c)=3, =4, and =5 by changing the loading time t_{L} through adjustment of a soft cushion spring constant. For the rapid load test on this occasion, dynamic signal (load, acceleration) measurements were made only at the pile head.

(1) Result of Analysis with Unloading Point (ULP) Method

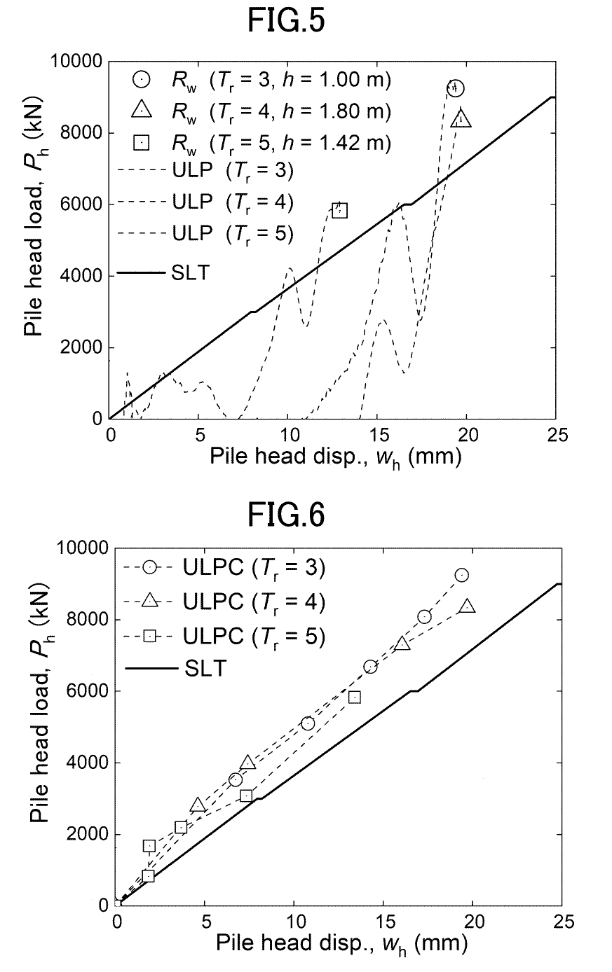

FIG. 5 shows a comparison between a pile head load Pₕ-pile head displacement w relation obtained from analysis with the Unloading Point (ULP) method and the static load test result, under consideration of the rapid load tests conducted respectively when Tᵣ=3, =4, and =5.

For the analysis of each rapid load test, the dynamic signals caused by application of a single blow were used to calculate the Pₕ−w relation after determination of a damping constant C. As a result, it is found that there is a wide divergence between the Pₕ−w relation obtained from the Unloading Point (ULP) method using the dynamic signals caused by only the application of the single blow and the static load test result.

(2) Result of Analysis with Unloading Point Connection (ULPC) Method

FIG. 6 shows a comparison between the Pₕ−w relation obtained from analysis using the Unloading Point Connection (ULPC) method and the static load test result, considering the rapid load tests conducted at Tᵣ=3, 4, and 5.

In comparison with the result shown in FIG. 5, it is found that the result of analysis with the Unloading Point Connection (ULPC) method can analogously express the static load test result. However, it is also found that at certain displacements w of the pile, overestimation of Pₕ appears with shortening of Tᵣ.

(3) Result of Analysis with Analysis Method (ULPC_CM Method) According to the Present Invention

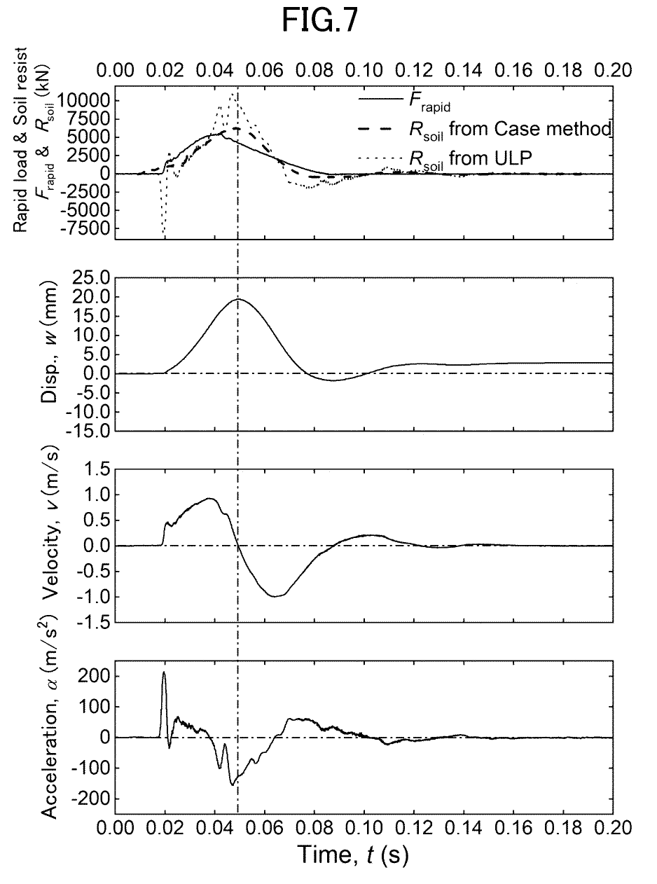

FIG. 7 shows time histories of rapid load F_{rapid}, displacement w, velocity v, and acceleration α in the rapid load test conducted when Tᵣ=3 and the weight drop height h=1.0 m. It is to be noted that in FIG. 7, R_{soil(Case)} obtained from the Case method and R_{soil(ULP)} obtained from the Unloading Point (ULP) method are shown along with F_{rapid}.

As of the maximum displacement of the pile, the velocity vis 0. Thus, R_{soil(Case)}, as of the maximum displacement of the pile, shall be defined to be static resistance R_{w} (R_{ULP}); likewise, the Unloading Point (ULP) method. Accordingly, the static load-displacement relation is constructed by connecting the unloading point load and the displacement that are respectively obtained with the analysis method of the present invention from a series of rapid load tests conducted in the manner of changing the weight drop height h.

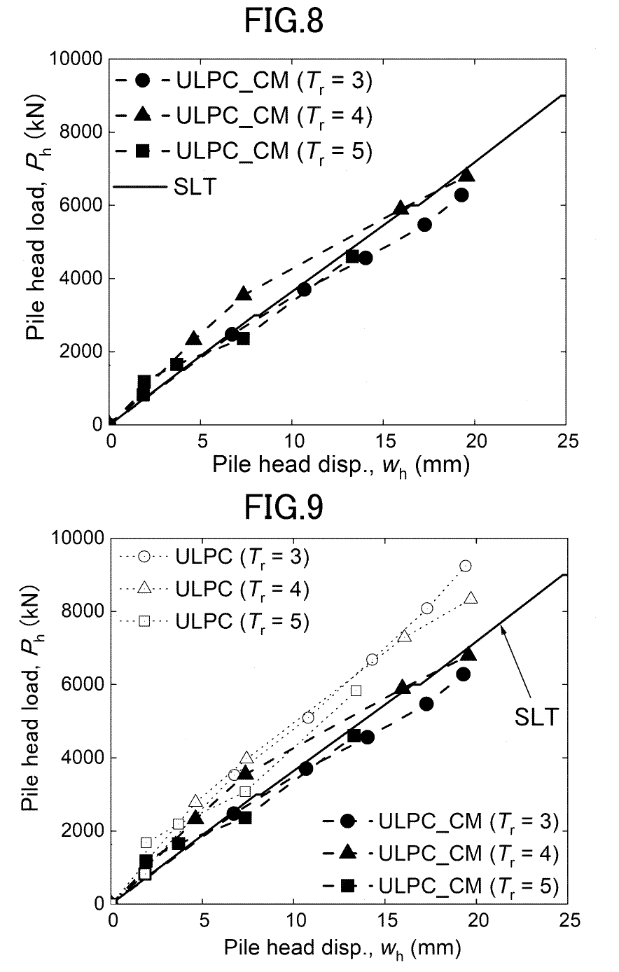

FIG. 8 shows a comparison between the Pₕ−w relation obtained using the analysis method of the present invention and the static load test result, considering the rapid load tests conducted at Tᵣ=3, 4, and 5.

Even for any of the rapid load tests, it is found that the result of analysis with the analysis method of the present invention is approximately in agreement with the static load test result. In other words, it can be appreciated that for the analysis method of the present invention, the Pₕ−w relation remains nearly unaffected by Tᵣ, even in the case of Tᵣ shortened to 3.

It is to be noted that the results shown in FIGS. 6 and 8 are given altogether in FIG. 9 to make it easy to compare the result of analysis with the analysis method of the present invention and that with the Unloading Point Connection (ULPC) method.

### (4) Weighing of Analysis Results

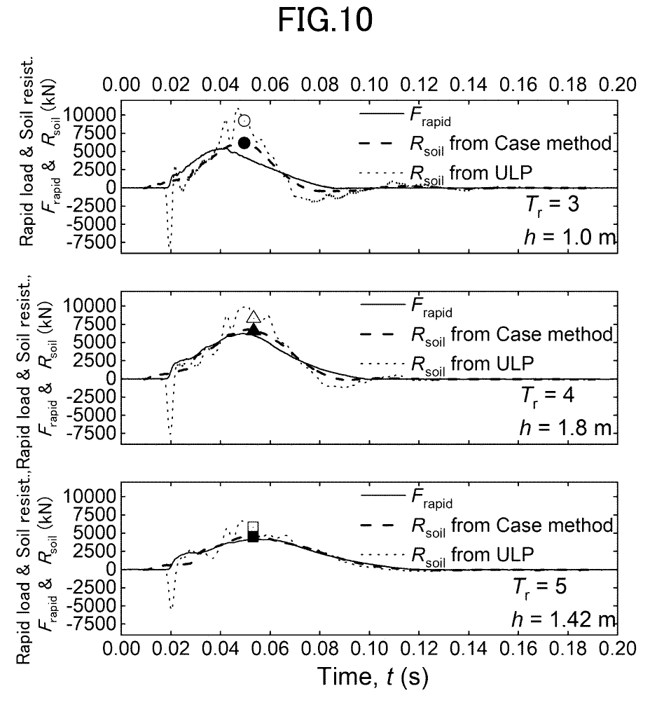

FIG. 10 shows time histories of F_{rapid}, R_{soil(Case)}, and R_{soil(ULP)} in the rapid load tests conducted respectively when Tᵣ=3, =4, and =5. It is found that there is not much of a difference between R_{soil(Case)} and R_{soil(ULP)} when Tᵣ=5.

However, it is also found that a more significant difference appears between R_{soil(Case)} and R_{soil(ULP)} as Tᵣ is shortened from 4 to 3. For the rapid load test results on this occasion, the analysis with the Unloading Point Connection (ULPC) method has been limited in application thereof to the rapid load test conducted when Tᵣ=5. On the contrary, the analysis with the analysis method according to the present invention has proved to be applicable up to the rapid load test conducted when Tᵣ=3.

Consequently, it can be appreciated that according to the rapid load test method for the pile/the analysis method of the present invention makes it possible to determine a more highly precise load-displacement relation.

1. A rapid load test method for a pile, in which a weight is dropped onto a pile head to determine a static load-displacement relation of the pile from a relation between a load caused by the drop of said weight and a displacement of said pile head, wherein the rapid load test method comprises:
conducting a plurality of rapid load tests on said pile head in the manner of changing the drop height h of said weight,
determining soil resistance Rₛₒᵢₗ to the pile under receiving a blow, in order of the following procedures (a) and (b) according to a Case method based on one-dimensional wave propagation theory, for each of the plurality of rapid load tests,
determining a relation between a displacement w of said pile head and said soil resistance Rₛₒᵢₗ from temporal changes of the directly measured displacement w of said pile head,
determining an unloading point load Pₕ and an unloading point displacement wₕ by assuming that the soil resistance Rₛₒᵢₗ₍ₘₐₓ₎ as of the maximum displacement wₘₐₓ is being static resistance R_{w}, and
determining a pile head load Pₕ-pile head displacement wₕ relation resulting from connecting said unloading point load Pₕ and said unloading point displacement wₕ, respectively obtained for each different drop height h,
(a) calculating a downward traveling wave F_{d} and an upward traveling wave Fᵤ from axial force F and pile velocity v in the rapid load test by using the following equations (5) and (6),
$$\begin{matrix}[ Equation 5 ] & \\ {F}_{d} ( {x}_{m} , t ) = \frac{F ( {x}_{m} , t ) + Z v ( {x}_{m} , t )}{2} & ( 5 )\end{matrix}$$
$$\begin{matrix}[ Equation 6 ] & \\ {F}_{u} ( {x}_{m} , t ) = \frac{F ( {x}_{m} , t ) - Z v ( {x}_{m} , t )}{2} & ( 6 )\end{matrix}$$
(b) Subsequently, obtaining the temporal changes of the soil resistance Rₛₒᵢₗ to the pile by using the following equation (7),
$$\begin{matrix}[ Equation 7 ] & \\ {R}_{soil} ( {x}_{m} , t ) = {F}_{d} ( {x}_{m} , t - \frac{{L}_{m}}{c} ) + {F}_{u} ( {x}_{m} , t + \frac{{L}_{m}}{c} ) & ( 7 )\end{matrix}$$
wherein x is coordinate along a pile axis (with the pile head set to be 0), xₘ is measurement position, v is pile velocity, Lₘ is length from the measurement position to a pile tip, F is axial force, F_{d} is downward traveling wave, Fᵤ is upward traveling wave, Z is impedance (EA/c) of a pile body, c is longitudinal wave velocity, E is Young's modulus of a pile material, A is cross-sectional area of the pile, and Rₛₒᵢₗ is soil resistance.
